(19) United States
(12) Patent Application Publication
(10) Pub. No.: US 2019/0092943 A1
(43) Pub. Date: Mar. 28, 2019

(54) SOLUTION OF POLYSULFONE IN N-ACYL-PYRROLIDINE AND USE THEREOF FOR UF MEMBRANES

(71) Applicant: BASF SE, Ludwigshafen am Rhein (DE)

(72) Inventors: Oliver GRONWALD, Ludwigshafen (DE); Martin WEBER, Ludwigshafen (DE); Ulrich KARL, Ludwigshafen (DE); Ulrich ABEL, EI Nido (PH); Thomas REISSNER, Ludwigshafen (DE); Nicolas MARION, Saint-Louis (FR)

(73) Assignee: BASF SE, Ludwigshafen am Rhein (DE)

(21) Appl. No.: 16/085,908

(22) PCT Filed: Mar. 8, 2017
(86) PCT No.: PCT/EP2017/055378

§ 371 Date: Sep. 17, 2018

(30) Foreign Application Priority Data
Mar. 18, 2016 (EP) 16161059.7

(51) Int. Cl. C08L 81/06; B01D 71/68; B01D 67/00; C07D 207/06

(52) U.S. Cl. CPC C08L 81/06; B01D 71/68; C08L 2201/56; C07D 207/06; B01D 67/0011

(57) ABSTRACT

Solution comprising a sulfone polymer and a N-acyl-pyrrolidine of formula I wherein R¹ to R⁹ independently from each other are a hydrogen atom or a methyl group. The solution is applied in membrane formation with polyvinylpyrrolidone as optional component.

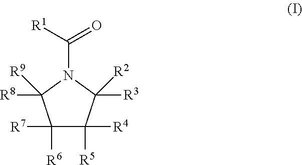

The present invention relates to a solution comprising a sulfone polymer and a N-acyl-pyrrolidine of formula I

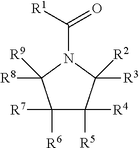

wherein R1 to R9 independently from each other are a hydrogen atom or a methyl group.

Sulfone polymers such as polysulfone, polyethersulfone and polyphenylsulfone are high performance polymers which are used in a variety of technical applications because of their mechanical properties and their chemical and thermal stability. Sulfone polymers, however, have limited solubility in many common solvents. In particular low molecular weight fractions of sulfone polymers cause turbidity of solutions of sulfone polymers, as described in J. G Wijmans and C. A. Smolders, Eur. Polym. J. 19, No. 12, pp 1143 to 1146 (1983). U.S. Pat. No. 5,885,456 discloses N-methylpyrrolidon (NMP), dimethylacetamide (DMAC), dimethylacrylamide (DMAD) or dimethyl-sulfoxide (DMSO) as suitable solvent for sulfone polymers.

One major technical application is the use of sulfone polymers as raw materials for the production of membranes, for example ultrafiltration membranes (UF membranes), as described in U.S. Pat. Nos. 4,207,182 and 5,885,456. The process of producing membranes of sulfone polymers includes dissolving sulfone polymers in a solvent, coagulating the sulfone polymer from such solvent and further post-treatment steps. The selection of the solvent is essential to the process and has impact on the properties of the obtained membrane, including but not limited to the membranes' mechanical stability, water permeability and minimum size of pores.

N-formyl-pyrrolidine and N-acetyl-pyrrolidine are chemical compounds that are liquid at room temperature. Their use as solvents is known from U.S. Pat. No. 2,404,719 relating to poly-acrylnitril solutions and from U.S. Pat. No. 5,173,112 relating to aqueous ink jet compositions. European patent application number 15182186.5 (PF 78779) relates to the use of N-formyl-pyrrolidine and N-acetyl-pyrrolidine as solvents for polyimide.

In the field of solvents there is an ongoing demand for alternative solvents which may replace presently used solvents in specific applications. In case of sulfone polymers alternative solvents should be able to prepare solutions that allow a high content of sulfone polymer without turbidity. Regarding membranes made there from it is important that at least the same standard of membrane quality and possibly an even better membrane quality is achieved. In particular, the water permeability of such membranes should be as high as possible and the pore size should be low.

It was an object of the present invention to provide an alternative solvent for sulfone polymers and for the process of making membranes. The alternative solvent should fulfill the requirements listed above.

Accordingly, the solution as defined above and a process for the making of membranes have been found.

To the Sulfone Polymer

The solution comprises a sulfone polymer. The term “sulfone polymer” shall include a mixture of different sulfone polymers.

A sulfone polymer comprises —SO₂— units in the polymer, preferably in the main chain of the polymer.

Preferably, the sulfone polymer comprises at least 0.02 mol —SO₂— units, in particular at least 0.05 mol —SO₂— units per 100 grams (g) of polymer. More preferred is a sulfone polymer comprising at least 0.1 mol —SO₂— units per 100 g of polymer. Most preferred is a sulfone polymer comprising at least 0.15 SO₂— units, in particular at least 0.2 mol —SO₂— units per 100 g of polymer.

Usually a sulfone polymer does comprise at maximum 2 mols —SO₂— units, in particular at maximum 1.5 mols of —SO₂— units per 100 grams (g) of polymer. More preferred is a sulfone polymer comprising at maximum 1 mol and of —SO₂— units per 100 grams of polymer. Most preferred is a sulfone polymer comprising at maximum 0.5 of —SO₂— units per 100 grams of polymer.

Preferably, the sulfone polymer comprises aromatic groups, shortly referred to as an aromatic sulfone polymer.

In a preferred embodiment, the sulfone polymer is an aromatic sulfone polymer, which consists to at least 20% by, in particular to at least 30% weight of aromatic carbon atoms. An aromatic carbon atom is a carbon atom, which is part of an aromatic ring system.

More preferred is an aromatic sulfone polymer, which consists to at least 40% by weight, in particular to at least 45% by weight of aromatic carbon atoms.

Most preferred is an aromatic sulfone polymer, which consists to at least 50% by weight, in particular to at least 55% by weight of aromatic carbon atoms.

Preferably, the sulfone polymer may comprise aromatic groups that are selected from 1,4-phenylen, 1,3-phenylene, 1,2-phenylene, 4,4′-biphenylene, 1,4-naphthylene, or 3-chloro-1,4-phenylene.

The aromatic groups may be linked by, for example, units selected from —SO₂—, —SO—, —S—, —O—, —CH₂—, —C(CH₃)₂.

In a preferred embodiment, the sulfone polymer consists to at least 80% by weight, more preferably to at least about 90% by weight and most preferably to at least 95, respectively at least 98% by weight of groups selected from the above aromatic groups and linking groups.

Examples of most preferred sulfone polymers are:

polyethersulfone of formula II

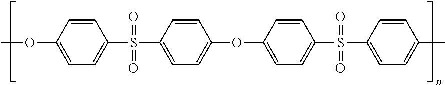

which is, for example, available from BASF under the trade name Ultrason® E,

polysulfone of formula III

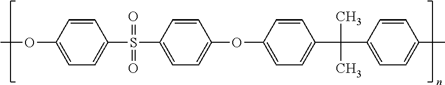

which is, for example, available from BASF under the trade name Ultrason® S and

polyphenylsulfone of formula IV

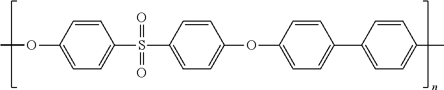

which is, for example, available from BASF under the trade name Ultrason® P.

to the N-acyl-pyrrolidine

R1 to R9 in formula I independently from each other are a hydrogen atom or a methyl group.

Preferably, at least 5 groups, in particular at least 7 groups of groups R² to R⁹ are hydrogen.

More preferably all groups R² to R⁹ are hydrogen.

Hence in such more preferred embodiment the N-acyl-pyrrolidine is N-formyl-pyrrolidine (R¹ to R⁹ are hydrogen) or N-acetyl-pyrrolidine (R¹=methyl, R² to R⁹ are hydrogen).

In a most preferred embodiment R¹ is a hydrogen and the N-acyl-pyrrolidine is N-formyl-pyrrolidine.

The term “N-acyl-pyrrolidine” as used herein shall include also a mixture of N-acyl-pyrrolidines.

To the Solution

The solution may comprise further solvents besides the N-acyl-pyrrolidine of formula I, hereinafter referred to as co-solvents.

Preferred are co-solvents that are miscible with the N-acyl-pyrrolidine in any ratio. Suitable co-solvents are, for example, N-methylpyrrolidone (NMP), N-ethylpyrrolidon (NEP), dimethylacetamide (DMAc), dimethylformamide (DMF), dimethylacrylamide (DMAD), dimethylsulfoxide (DMSO) or alkylencaronates as such as in particular propylene carbonate.

In a preferred embodiment at least 30% by weight, in particular at least 50% by weight by weight of the total amount of all solvents of the solution is N-acyl-pyrrolidine.

In a more preferred embodiment at least 70% by weight, in particular at least 90% by weight by weight of the total amount of all solvents of the solution is N-acyl-pyrrolidine.

In a most preferred embodiment at least 95% by weight, in particular at least 98% by weight by weight of the total amount of all solvents of the solution is N-acyl-pyrrolidine.

In a most preferred no co-solvent is used in the solution and N-acyl-pyrrolidine of formula I is the only solvent used.

Preferably, the solution comprises 5 to 200 parts by weight, in particular 10 to 100 parts, more preferably 15 to 50 parts by weight of sulfone polymer per 100 parts by weight of the total amount of all solvents.

In a most preferred embodiment the solution comprises 5 to 200 parts by weight, in particular 10 to 100 parts, more preferably 15 to 50 parts by weight of sulfone polymer per 100 parts by weight of the total amount of N-acyl-pyrrolidine of formula I.

The solution may be prepared by adding the sulfone polymer to the solvent and dissolving the polysulfone according to any process known in the art. The dissolution process may be supported by increasing the temperature of the solution and/or by mechanical operations like stirring. In an alternative embodiment the sulfone polymer may be already synthesized in N-acyl-pyrrolidine or a solvent mixture comprising N-acyl-pyrolidine.

To the Process of Making a Membrane

In the context of this application a membrane shall be understood to be a semipermeable structure capable of separating two fluids or separating molecular and/or ionic components or particles from a liquid. A membrane acts as a selective barrier, allowing some particles, substances or chemicals to pass through, while retaining others. The membrane may have various geometries such as flat sheet, spiral wound, pillows, tubular, single bore hollow fiber or multiple bore hollow fiber.

For example, membranes can be reverse osmosis (RO) membranes, forward osmosis (FO) membranes, nanofiltration (NF) membranes, ultrafiltration (UF) membranes or microfiltration (MF) membranes. These membrane types are generally known in the art and are in detail described in literature. A good overview is found also in earlier European patent application No. 15185604.4 (PF 78652) which is here with incorporated herein by reference. A preferred membrane is the ultrafiltration (UF) membrane.

Membranes may be produced according to a process comprising the following steps:
a) providing a solution comprising a sulfone polymer and N-acyl-pyrrolidine of formula I and further comprising a water soluble polymer,
b) contacting the solution with a coagulant
c) optionally oxidizing and washing the obtained membrane

The solution in step a) corresponds to the solution described above. To this solution a water soluble polymer is added. The water soluble polymer helps to adjust the viscosity of the solution. The main purpose of the water solution polymer is to support the formation of the pores. In the following coagulation step b) the water soluble polymer becomes distributed in the coagulated membrane and thus becomes the place holder for pores.

The water soluble polymer may be any known water soluble polymer. As examples polyvinyl pyrrolidones or polyalkylene oxides like polyethylene oxides may be mentioned. A preferred water soluble polymer is polyvinyl pyrrolidone.

In a preferred embodiment, the solution in step a) comprises 99.9 to 50% by weight of the sulfone polymer and 0.1 to 50% by weight of the water soluble polymer, based on the total weight of the sulfone polymer and water soluble polymer.

Preferably, the solution comprises 95 to 70% by weight of the sulfone polymer and 5 to 30% by weight of the water soluble polymer based on the total weight of the sulfone polymer and water soluble polymer.

The solution may optionally be degassed before proceeding to the next step.

In step b) the solution is contacted with a coagulant. In this step coagulation of the sulfone polymer occurs and the membrane structure is formed.

The sulfone polymer should have low solubility in the coagulant. Suitable coagulants are, for example, liquid water, water vapor, alcohols or mixtures thereof.

Suitable alcohols are, for example, mono-, di- or trialkanols like iso-propanol, ethylene glycol or propylene glycol. A preferred coagulant is liquid water.

Further details of process steps a) and b) depend on the desired geometrical structure of the membrane and the scale of production, which includes lab scale or commercial scale.

For a flat sheet membrane detailed process steps a) and b) could be as follows:
a1) adding the water soluble polymer to the solution comprising a sulfone polymer and N-acyl-pyrrolidine of formula I

a2) heating the solution until a viscous solution is obtained; typically the solution is kept at a temperature of 5-250° C., preferably 25-150° C., more preferably 50-90° C.

a3) further stirring of the solution until a homogenous mixture is formed; typically homogenization is finalized within 1-15 h, preferably within 0.5 to 3 hours

b1) Casting the solution obtained in a3) on a support and thereafter transferring the casted film into a coagulation bath, which is preferably water.

For the production of single bore hollow fiber or multiple bore hollow fibers step b1) may performed by extruding the solution obtained in a3) through an extrusion nozzle with the required number of hollow needles. The coagulating liquid is injected through the hollow needles into the extruded polymer during extrusion, so that parallel continuous channels extending in extrusion direction are formed in the extruded polymer. Preferably the pore size on an outer surface of the extruded membrane is controlled by bringing the outer surface after leaving the extrusion nozzle in contact with a mild coagulation agent such that the shape is fixed without active layer on the outer surface and subsequently the membrane is brought into contact with a strong coagulation agent.

Further process step c) is optional. In a preferred embodiment process step c) is performed. Oxidation as well as washing is performed in order to remove the water soluble polymer and to form the pores. Oxidation may be followed by washing or vice versa. Oxidation and washing may as well be performed simultaneously in one step.

For oxidation any oxidant may be used. Preferred is a water soluble oxidant such as in particular sodium hypochlorite.

According to the invention solutions of sulfone polymer are obtained that show no or at least less turbidity. The solutions are suitably for the manufacturing of membranes. Membranes obtained have high mechanical stability and have excellent separation characteristics. In particular, membranes have very good molecular weight cutoffs (MWCO). In a preferred embodiment, membranes M have a molecular weight cutoff, determined as described in the experimental section, of less than 20 kDa (20.000g/mol). The membranes further have very good water permeabilities. In a preferred embodiment, membranes have a pure water permeability (PWP), determined as described in the experimental section, of at least 300 kg/h m² bar, in particular of at least 500 kg/h m² bar.

The membranes obtained by the process of the invention may be used for any separation purpose, for example water treatment applications, treatment of industrial or municipal waste water, desalination of sea or brackish water, dialysis, plasmolysis, food processing.

### EXAMPLES

Abbreviations and compounds used in the examples:

DMAc Dimethylacetamide

PWP pure water permeation

MWCO molecular weight cutoff

NFP N-formylpyrrolidine
Ultrason® S 6010 Polysulfone with a viscosity number (ISO 307, 1157, 1628; in 0.01 g/mol phenol/1,2 orthodichlorobenzene 1:1 solution) of 81; a glass transition temperature (DSC, 10° C./min; according to ISO 11357-1/-2) of 187° C.; a molecular weight Mw (GPC in THF, PS standard): 60000 g/mol, Mw/Mn=3.7
Luvitec® K90 Polyvinylpyrrolidone with a solution viscosity characterised by the K-value of 90, determined according to the method of Fikentscher (Fikentscher, Cellulosechemie 13, 1932 (58))

The pure water permeation (PWP) of the membranes was tested using a pressure cell with a diameter of 60 mm using ultrapure water (salt-free water, filtered by a Millipore UF-system). A high PWP allows a high flow rate and is desired.

In a subsequent test, solutions of PEG-Standards with increasing molecular weight were used as feed to be filtered by the membrane at a pressure of 0.15 bar. By GPC-measurement of the feed and permeate, the molecular weight of the permeate of each PEG-Standard used was determined. The weight average molecular weight (MW) cut-off of the membranes (MWCO) is the molecular weight of the first PEG Standard which is withhold to at least 90% by the membrane. For example a MWCO of 18400 means that PEG of molecular weight of 18400 and higher are withhold to at least 90%. It is desired to have a lowest MWCO as possible.

Preparation of Membranes Using NFP as Polymer Solvent

General Procedure

Into a three neck flask equipped with a magnetic stirrer there were added 75 ml of Solvent S1 as given in table 1, Luvitec K90 (“K90”) as second dope polymers with the amounts given in table 1 and 19 g of polysulfone (Ultrason® S 6010). The mixture was heated under gentle stirring at 60° C. until a homogeneous clear viscous solution, usually referred to as dope solution was obtained. The solution was degassed overnight at room temperature.

In table 1 results are listed for DMAc (comparative) and NFP (example).

TABLE 1
Properties of the dope polymer solutions
based on different solvents S1.

| | K90 | Solvent S1 | appearance dope solution |
|---|---|---|---|
| Comparative | 6 | DMAc | turbid |
| Example | 6 | NFP | clear and transparent |

After that the membrane solution was reheated at 60° C. for 2 hours and casted onto a glass plate with a casting knife (300 microns) at 60° C. using an Erichsen Coating machine operating at a speed of 5 mm/min. The membrane film was allowed to rest for 30 seconds before immersion in a water bath at 25° C. for 10 minutes. After the membrane had detached from the glass plate, the membrane was carefully transferred into a water bath for 12 h. Afterwards the membrane was transferred into a bath containing 2500 ppm NaOCl at 50° C. for 4.5 h. The membrane was then washed with water at 60° C. and one time with a 0.5 wt.-% solution of sodium bisulfite to remove active chlorine. After several washing steps with water the membrane was stored wet until characterization regarding pure water permeability (PWP) and minimum pore size (MWCO) started. Table 2 summarizes the membrane properties.

TABLE 2
Compositions and properties of membranes prepared;
MWCO in [Da], PWP in [kg/h m²bar].

| | K90 | Solvent S1 | PWP | MWCO |
|---|---|---|---|---|
| Comparative | 6 | DMAc | 530 | 30700 |
| Example | 6 | NFP | 730 | 18400 |

Membranes produced with NFP according to the invention show improved separation characteristics over membranes known from the art. Membranes produced with NFP show improved (smaller) MWCO in combination with higher permeability values compared to membranes known from the art.

1. A solution comprising:
a sulfone polymer;
and a N-acyl-pyrrolidine of formula (I): 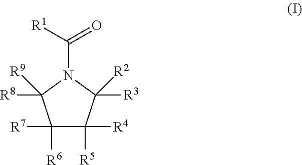
wherein R¹ to R⁹ are each independently selected from the group consisting of a hydrogen atom and a methyl group.

2. The solution according to claim 1, wherein the sulfone polymer comprises at least 0.02 mol SO₂^{—} units per 100 g of sulfone polymer.

3. The solution according to claim 1, wherein the sulfone polymer is an aromatic sulfone polymer, comprising at least 30% by weight of aromatic carbon atoms, based on a total weight of the sulfone polymer.

4. The solution according to claim 1, wherein the N-acyl-pyrrolidine of formula (I) is N-formyl-pyrrolidine or N-acetyl-pyrrolidine.

5. The solution according to claim 1, wherein the solution comprises 5 to 200 parts by weight of the sulfone polymer per 100 parts by weight of the N-acyl-pyrrolidine of formula (I).

6. A process for making the solution of claim 1, the process comprising dissolving or synthesizing the sulfone polymer in the N-acyl-pyrrolidine of formula (I).

7. A process for making a membrane, the process comprising:
a) mixing the solution according to claim 1 with a water-soluble polymer to obtain a second solution;
b) contacting the second solution with a coagulant to obtain a membrane; and
c) optionally oxidizing and washing the obtained membrane.

8. The process according to claim 7, wherein the water-soluble polymer in a) is polyvinylpyrrolidone.

9. The process according to claim 7, wherein the coagulant in b) is water.

10. A membrane obtained by the process according to claim 7.

11. A separation article for water treatment applications, treatment of industrial or municipal waste water, desalination of sea or brackish water, dialysis, plasmolysis, or food processing, the article comprising the membrane according to claim 10.
